(19) United States
(12) Patent Application Publication
(10) Pub. No.: US 2004/0028561 A1
(43) Pub. Date: Feb. 12, 2004

(54) System for the detection of pathogens in the mail stream

(75) Inventors: David W. Daugherty, Bixby, OK (US); William R. Harris, Jacksonville, FL (US); Timothy M. Herron, Endicott, NY (US); Irene M. Siedlarczyk, Johnson City, NY (US)

Correspondence Address: PERKINS, SMITH & COHEN LLP, ONE BEACON STREET, 30TH FLOOR, BOSTON, MA, 02108, US

(73) Assignee: Lockheed Martin Corporation

(21) Appl. No.: 10/291,013

(22) Filed: Nov. 8, 2002

Related U.S. Application Data
(60) Provisional application No. 60/348,211, filed on Nov. 9, 2001.
(60) Provisional application No. 60/344,846, filed on Dec. 31, 2001.

(51) Int. Cl. G01N 33/00; G01N 1/18

(52) U.S. Cl. 422/99; 422/73; 436/177

(57) ABSTRACT

The system and method of the present invention detect bio-hazardous-sized particles in, on, and around mail. The system collects these bio-hazardous-sized particles, tests for the presence of bio-hazardous particles, generates a signal when bio-hazardous particles are detected, and prepares an analysis sample and performs real-time or near real-time analysis and optionally air treatment.

### CROSS REFERENCE TO RELATED APPLICATIONS

The present application claims priority to U.S. Provisional Application No. 60/348,211 filed Nov. 9, 2001, entitled A BIOMAIL SYSTEM FOR THE DETECTION OF PATHOGENS and U.S. Provisional Application No. 60/344,846 filed Dec. 31, 2001, entitled A BIOMAIL SYSTEM FOR THE DETECTION OF PATHOGENS which are incorporated herein by reference.

### BACKGROUND OF THE INVENTION

This invention relates generally to the detection, containment, filtration, sampling, analysis, and decontamination of the mail stream from biological pathogens in the incoming, outgoing, and mail processing industry.

In federal and commercial incoming and outgoing mailroom operations, equipment and personnel are involved in the sortation, transport, and opening of actual mail pieces. In the course of these normal daily activities, chemical and biological agents have been inadvertently introduced into the mail operations environment as well as cross-contaminating previously non-contaminated mail pieces from pieces of mail maliciously doped with biological pathogens. These occurrences have contaminated equipment and personnel, and have caused increased operating costs and delays in mail service and deliveries. Also, potential lethal health risks have been introduced and have even caused some deaths in mail operation personnel, mail delivery personnel, and innocent end mail recipients and individuals.

Clearly the current mail handling and screening systems are deficient in detecting biological pathogens. In particular, the current systems are deficient in fully integrating biological filtration, detection, sampling, analysis, and decontamination from beginning to end of the mail handling process. Stand-alone equipment and technology are not sufficient to prevent biohazards from invading the ambient air in mail operations environments. Systems implemented for battlefield and warfare applications, where biological and chemical events are typically experienced in relatively low concentrations over relatively long periods of time, are not appropriate for mailroom applications. Unlike a battlefield situation, in a mail processing facility, high concentrations of contaminants delivered over a short period of time are more typical and existing technology is required to be modified or new technology needs to be created to account for these operational differences.

With the current biological and chemical threats being made against the U.S. Federal Government, the U.S. Postal Service and various commercial incoming and outgoing mail operations, a fully integrated end-to-end solution needs to be developed that will detect, filtrate, sample, analyze, and decontaminate the actual mail stream, the mail processing and transportation equipment and the re-circulated airflow in the ventilation systems. The solution should protect the associated operators and personnel from potentially harmful chemical and biological agents. A system is needed in which biohazards are detected and removed from the mail and the ventilation systems before contamination of the ambient workspace has occurred.

### SUMMARY OF THE INVENTION

The problems set forth above as well as further and other problems are solved by the present invention. The solutions and advantages of the present invention are achieved by the illustrative embodiment of the present invention described hereinbelow.

The bio-hazard collection and testing system of the present invention includes a collection subsystem for collecting particles in, on and around mail, a filtration subsystem connected to the collection subsystem for separating bio-hazardous-sized particles from collected particles for testing and for capturing the collected particles and the biohazardous-sized particles. The system also includes a triggering subsystem connected to the filtration subsystem for receiving and testing the bio-hazardous-sized particles to determine whether they are bio-hazardous particles, and generating a signal when biohazardous particles are detected. The system also includes a sampling subsystem connected to the triggering subsystem for preparing an analysis sample containing the bio-hazardous particles. The sampling subsystem prepares an analysis sample that includes the sampled bio-hazardous particles. The system of the present invention can optionally include an analysis subsystem for determining the composition of the biohazardous particles in the analysis sample. Furthermore, the system can optionally include an enclosure partially surrounding the collection subsystem for containing the bio-hazardous particles, i.e. for creating a barrier between the bio-hazardous particles and ambient air. The system can also optionally include a decontamination subsystem, connected to the filtration subsystem, for neutralizing the bio-hazardous particles. As used herein, the term “neutralizing” refers to deactivating, degrading, rendering substantially harmless, decontaminating, and/or sterilizing any hazardous agent detected. For example, if a bio-hazard, such as anthrax, is detected, “neutralizing” means treating it so that it is not a substantial, or any, risk to people, such as by subjecting the anthrax to chlorine dioxide. In the event the hazard is of a chemical nature, “neutralizing” means treating the hazardous agent so that the chemical is not a substantial, or any, risk to people. This treatment may be a gas, or any other substance that renders the hazardous material substantially safe to people.

The collection subsystem of the present invention collects particles that are hosted by objects, such as mail pieces, or particles that can be resident in the air surrounding the objects. The collection subsystem of the illustrative embodiment captures particles in a filtration subsystem. Depending upon the embodiment, the system can optionally include exhaust mechanism, a downdraft mechanism, or a ventilation subsystem, all possibly useful, in combination or separately, for directing particles from the objects and their surroundings to the filtration subsystem. The exhaust subsystem, when included, forces collected particles and bio-hazardous particles through the filtration system. Optionally, the system can include a transport subsystem for moving objects past the collection subsystem.

The ventilation subsystem can provide airflow communication among the collection subsystem, the triggering subsystem, and the exhaust subsystem. The ventilation subsystem can also move the collected particles and the bio-hazardous particles from the collection subsystem to the exhaust subsystem.

The triggering subsystem of the present invention generates a signal and activates the sampling subsystem if bio-hazardous particles, for example biological particles or chemical particles, are detected within the sample. The triggering subsystem continuously collects a sample of air containing particles. The triggering subsystem can also disable the transport subsystem, if present, when it detects bio-hazardous particles, but ensures that the filtration subsystem continues to operate after bio-hazardous particles are detected to ensure maximum contaminant containment. The triggering subsystem can also include a means for notifying authorities when bio-hazardous particles are detected.

The sampling subsystem of the present invention prepares a liquid medium based analysis sample of particle-containing air for real-time and/or off-line analysis. An optional automatic or manual analysis subsystem analyzes the particles to determine the nature of the particles. If the analysis subsystem executes in real-time or near real-time, the sampling subsystem can invoke a decontamination subsystem to neutralize the particles

The workstation embodiment includes mail screening workstation and mail opening workstation embodiments, either of which is a self-contained unit containing the components described in the illustrative embodiment above or a unit that is in airflow communication with a system of the illustrative embodiment described above. Objects such as parcels or mail pieces that must be processed by hand enter the mail opening workstation embodiment for manual processing. The downdraft subsystem of the collection subsystem continuously draws a stream of air away from the mail pieces to move particles away from the operator and in the direction of the collection subsystem. Objects such as mail pieces that can be automatically processed, i.e. letters and flats, enter the mail screening workstation embodiment, and the mail screening workstation operates substantially the same as the mail opening workstation.

The portable detection embodiment of the present invention can include a handheld device that collects air samples, filters the air, and prepares samples for either real-time or off-line analysis. The handheld device can be a conventional bio-hazardous particle detection device that prepares analysis samples for near real-time or post collection technologies. The handheld device can include a handheld collection subsystem for collecting particles from ambient air, a handheld filtration subsystem for capturing collected particles, and a handheld sampling subsystem for preparing a sample bio-hazardous particles in near real-time for later analysis.

The method of the present invention for detecting and isolating bio-hazardous particles in and around objects includes the steps of collecting particles in and around objects, filtering particles for size and concentration, testing the particles for the presence of bio-hazardous particles, and generating a signal when bio-hazardous particles are detected. The method of the present invention optionally includes the steps of preparing an analysis sample including the bio-hazardous particles, and preventing the bio-hazardous particles from contaminating the operational surroundings when bio-hazardous particles are detected. The method can optionally further include the step of determining the nature of the analysis sample. The method can also optionally include the step of neutralizing the bio-hazardous particles.

For a better understanding of the present invention, together with other and further objects thereof, reference is made to the accompanying drawings and detailed description. The scope of the present invention is pointed out in the appended claims.

### BRIEF DESCRIPTION OF THE SEVERAL VIEWS OF THE DRAWING

FIG. 1 is a schematic block diagram of the components of the system of the illustrative embodiment of the present invention;

FIG. 2 is a schematic block diagram of the components of the system of the illustrative embodiment of the present invention further including an isolation mechanism in which part of the system is enclosed to contain contaminants;

FIGS. 3A and 3B are schematic block diagrams of the mail screening workstation embodiment and the mail opening workstation of the illustrative embodiment of the present invention;

FIG. 3C is a schematic block diagram of the handheld sampling system of the illustrative embodiment of the present invention;

FIG. 4 is a flowchart of the method of the present invention;

FIG. 5 is a monitoring system configuration for local, regional and national monitoring of events generated by and from the system of the present invention;

FIG. 6A is an overhead view of the mail transport system of the illustrative embodiment of the present invention;

FIG. 6B is a component cross section of the mail opening workstation and the mail screening workstation of the illustrative embodiment of the present invention.

FIG. 7A is a schematic block airflow diagram of the illustrative embodiment of the present invention;

FIG. 7B illustrates a perpetual filter or scalper mechanism used to filter particles into the proper size range of interest; and

FIG. 8 is a block diagram example of a typical clean room environment in which the equipment of the present invention can be installed to further safeguard the mail operations.

### DETAILED DESCRIPTION OF THE INVENTION

The present invention is now described more fully hereinafter with reference to the accompanying drawings, in which the illustrative embodiment of the present invention is shown.

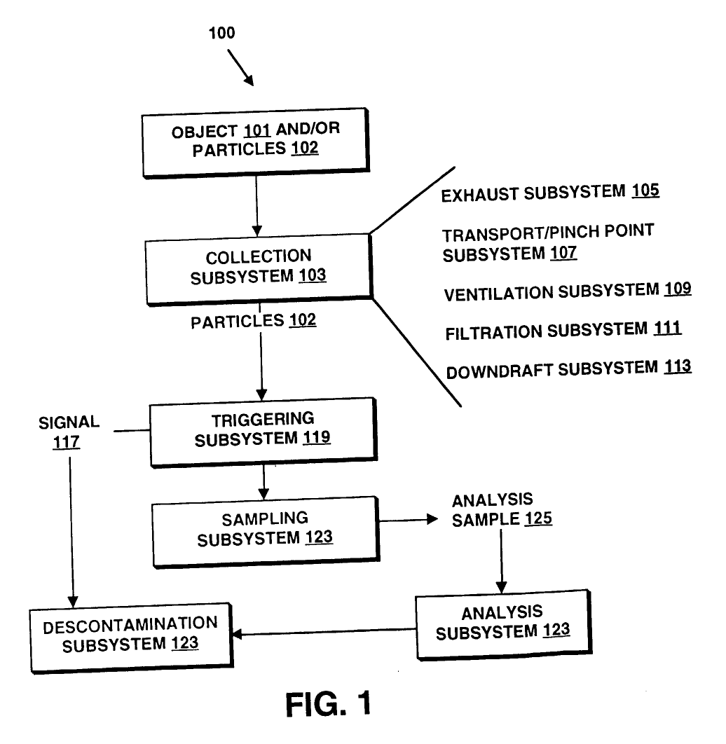

Referring now to FIG. 1, system 100 of the present invention includes collection subsystem 103 and triggering subsystem 119, and optionally sampling subsystem 123, analysis subsystem 127, and decontamination subsystem 115. Collection subsystem 103 can include, depending upon the embodiment of the invention, exhaust subsystem 105, transport/pinch point subsystem 107, ventilation subsystem 109, filtration subsystem 111, and/or downdraft subsystem 113.

Operationally, objects 101 laden with particles 102 and/or ambient particles either passively encounter collection subsystem 103 or are directed there by components of collection subsystem 103, or the particles 102 are forcefully ejected by transport/pinch point subsystem 107 such as pinch belt pulley assemblies that are normally installed in mail handling equipment to transport the mail from point to point depending upon the embodiment, and processing begins. Particles 102 are conveyed through a first component of filtration subsystem 111, the component that captures the correctly sized particles for further analysis but allows other particles to pass through the system and ultimately encounter the second component of filtration subsystem 111, the component that traps remaining particles in the air before the air is exhausted from the system. Triggering subsystem 119 samples particles 102 from filtration subsystem 103 and generates a signal 117 if bio-hazardous particles are detected. Optionally triggering subsystem activates the sampling subsystem 123 if bio-hazardous particles are detected. The sampling subsystem 123 prepares an analysis sample 125 including the biohazardous particles. Analysis subsystem 127 can analyze the analysis sample 125 in near real-time or off-line later to determine the nature of the bio-hazardous particles present in the analysis sample 125. If bio-hazardous particles are detected, decontamination subsystem 115 can be activated to neutralize the bio-hazardous particles.

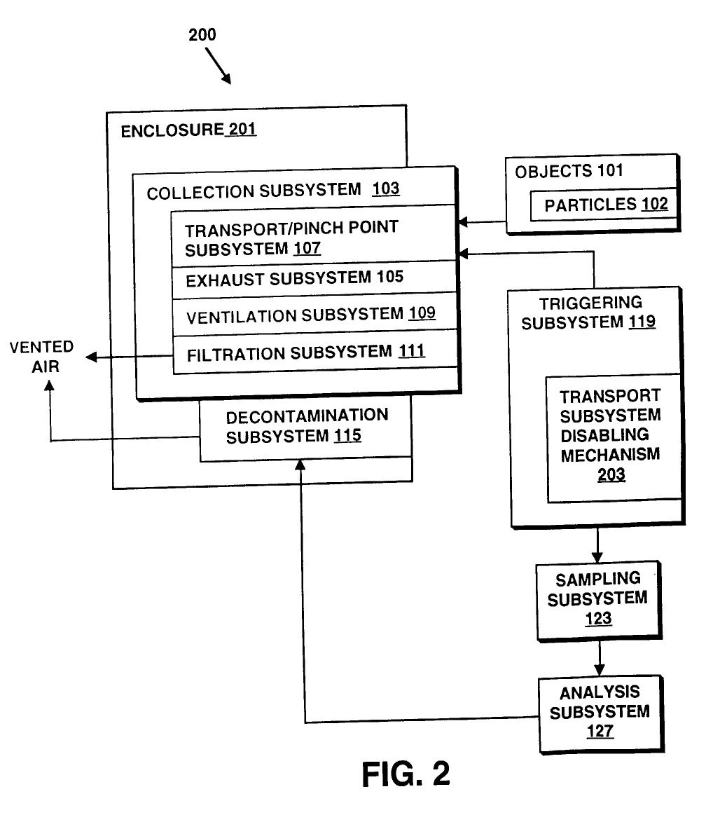

Referring now to FIG. 2, in the illustrative embodiment of the present invention, certain parts of the mail processing system are enclosed, for example with a ventilation hood or in a container, to prevent personnel and workplace contamination due to the presence of biological contaminants. Enclosure 201 attempts to isolate the air surrounding the collection subsystem 103 from air outside the enclosure, either physically and/or through continuous negative air pressure using the exhaust 105 and ventilation 109 subsystems, allowing for the intake of objects/particles 101/102 through transport/pinch point subsystem 107, for example a conventional conveyor belt, and venting of air beyond filtration subsystem 111. Enclosure 201 can also be used to isolate the decontamination subsystem 115. Within enclosure 201, in the illustrative embodiment of the present invention, negative air pressure set up by exhaust subsystem 105 causes air to be drawn into the ventilation subsystem 109 along with any particles 102 that may be generated or exist with the processing of objects 101, for example mail pieces. Negative air pressure results from the removal of air by exhaust subsystem 105 or other physical conditions, and subsequent replacement of air through make-up air intake. The flow resulting from the air exhaust/make-up condition has the effect of evacuating airborn particles, such as Bacillus anthracis spores, into the ventilation and filtration subsystems, 109 and 111, respectively, and away from the mail operators, equipment and mail operations environment.

Continuing to refer to FIG. 2, ventilation subsystem 109 conveys particles 102 to triggering subsystem and through filtration subsystem 111. Triggering subsystem 119 determines if the particles 102 are any sort of bio-hazardous particles, i.e. particles that could be biological, chemical, etc. If so, then the triggering subsystem 119 signals the sampling subsystem 123, which initiates the collection of a concentrated liquid medium based analysis sample 125 containing the bio-hazardous particles that can be used for near real-time and off-line analyses. The liquid medium analysis sample 125 can be analyzed in substantially real-time by analysis subsystem 127 and also possibly stored for offline post-forensic analysis. The transport subsystem disabling mechanism 203 can optionally disable power to the transport/pinch point subsystem 107 to minimize further contamination and cross-contamination, but it also ensures that the filtration subsystem 111 continues to operate to minimize the exposure of personnel and equipment to any contaminants of interest.

Referring to FIGS. 1 and 2, triggering subsystem 119 can concentrate air and particles, perhaps in a conventional aerosolizing concentrator, and for example, injects the air and particles into a conventional sampling chamber which can contain a conventional liquid source reservoir, conventional liquid collection vials, and a conventional liquid discharge collection reservoir. Sampling subsystem 123 prepares a liquid sample suitable for conventional bioassay test strip analysis, conventional polymerase chain reaction (PCR) analysis as well as conventional laboratory plating and culture growth.

Continuing to refer to FIGS. 1 and 2, exhaust subsystem 105 can include any conventional exhaust mechanism such as a fan. Ventilation subsystem 109 can include any conventional venting mechanism, active or passive, for moving air and particles from one space to another. Transport subsystem disabling mechanism 203 can include a signal-enabled switch that reacts to the signal generated by the triggering subsystem to reverse the orientation of the switch that controls the power to the transport subsystem 119.

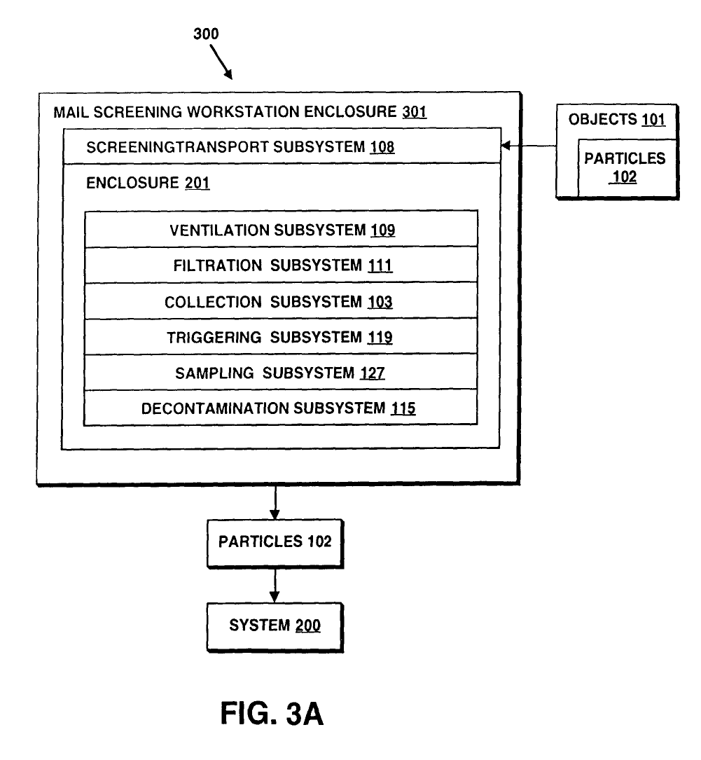

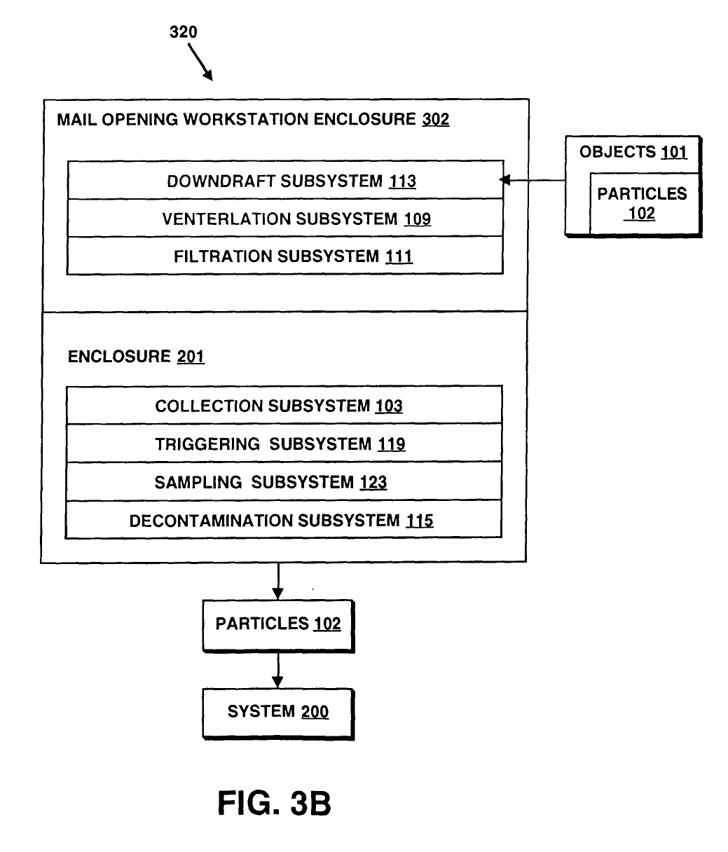

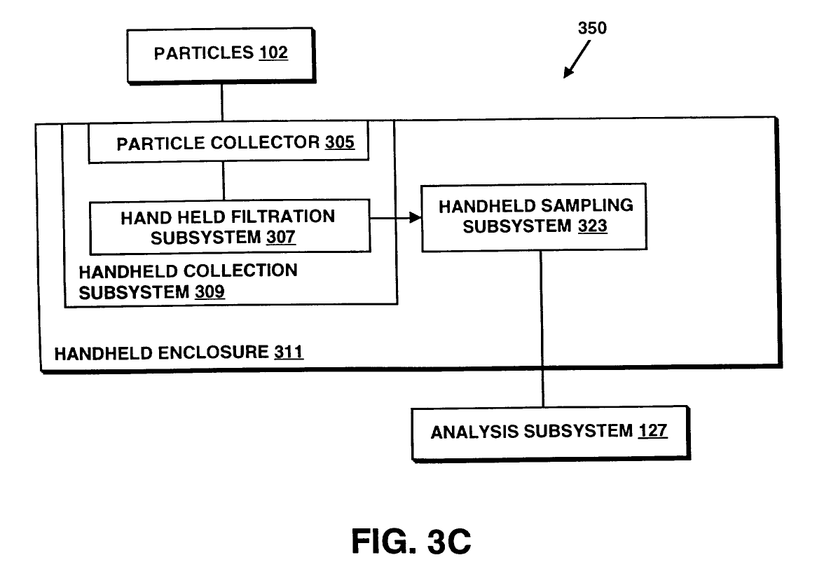

Referring now to FIGS. 3A and 3B, a workstation embodiment of the present invention can consist of a stand-alone mail screening workstation 300 or mail opening workstation 320 shown in FIGS. 3A and 3B respectively. Each system can include collection 103 and triggering 119, and perhaps sampling 123, screening transport 108, and decontamination 115 subsystems similar to system 100 and mail sortation system 200, respectively, of the illustrative embodiment of the present invention. Operationally, personnel place objects 101 hosting particles 102 into mail screening workstation enclosure 301 or mail opening workstation enclosure 302. Both mail screening workstation 300 and mail opening workstation 320 provide a downdraft subsystem 113 that can include a continuous downdraft of air onto, or in the alternative, negative air pressure around, objects 101. The mail screening workstation 300, however, provides for automated mail inspection, and thus the downdraft subsystem 113 is provided within enclosure 201 for the purpose of directing particles into the filtration subsystem 111. The mail opening workstation, on the other hand, is built for manual mail opening, and thus the downdraft subsystem 113 is located outside enclosure 201, and directs any particles that are dislodged from the mail away from personnel who are opening incoming mail, thus reducing the chance of aerosolizing any contaminants such as biological contaminants. The mail opening workstation enclosure 302 could be constructed such that a conventional drafting mechanism, such as a fan and blower assembly, blows air away from the position in which a worker would normally stand while inspecting mail. The collection 103 and filtration 111 subsystems of either the mail screening workstation 300 or the mail opening workstation 320 filter and collect particles 102 and optionally prepare them for sampling subsystem 123, as described above. Either mail screening workstation 300 or mail opening workstation 320 can optionally be integrated with a system such as mail sortation system 200 (shown in FIG. 2) of the present invention to vent and exhaust air containing particles 102 to mail sortation system 200. The mail screening workstation enclosure 301 and enclosure 201 can be containers manufactured from steel, plastic, or any other air-impermeable material.

Referring to FIGS. 1, 2, and 3A-B, the triggering subsystem 119, that continuously samples the particle-containing air, can provide the particle-containing air by pitot tubes, preferably at a low flow rate. Pitot tubes are optimally placed in the ventilation subsystem to provide the greatest probability of capturing the contaminant sample. Pitot tubes are small ventilation sampling tubes that are used to convey air samples from the main ventilation subsystem 109 to the triggering subsystem 119 and the sampling subsystem 123. The triggering subsystem 119 can distinguish between biological and non-biological particles, if those are the bio-hazardous-sized particles, through use of the properties of the ultraviolet (UV) light spectrum: natural fluorophores that exist in tissue and cells which, when excited with UV and visible light, fluoresce over well-defined spectral regions. The triggering subsystem also uses the concentration and size of the particles to differentiate biological particles from other particles. Thus, in the illustrative embodiment, triggering subsystem 119 can determine if particles are biological in nature through a conventional Laser or LED-based UV Fluorescence Particle Counter such as the PSI Bioni® Triggering Mechanism.

Continuing to refer to FIGS. 1, 2, and 3A-B, decontamination subsystem 115 can include a conventional Air Treatment System that can perform biological decontamination (among other types of decontamination) through use of UV energy that cause permanent deactivation of micro-organisms by disrupting DNA so that they are no longer able to maintain metabolism or reproduce. The decontamination subsystem 115 can also neutralize the air within the filtration subsystem 111 if necessary by injecting a chemical agent into the filtration subsystem 111. A conventional back draft damper can insure that the chemical agent will not back flow into the ventilation ductwork.

Continuing to refer to FIGS. 1, 2, and 3A-B, analysis subsystem 127 can include any system that can perform conventional analysis on conventional liquid medium based samples, for example PCR or DNA analysis, to determine the nature of the particles in the sample. PCR is a technique that enables the catalyzation of the formation and repair of DNA (and RNA). Target DNA can be exponentially amplified through this process, providing essentially unlimited quantities of the precise genetic material.

Referring now to FIG. 3C, portable detection embodiment 350 of the present invention is a portable air-sampling device that can be held, carried, worn, or mounted, and may be battery-operated. It may be worn by personnel, or placed strategically in or around equipment, similar in use to radiation detection badges, and used to take handheld samples of suspected contaminants. A portable particle collector 305 passively and continuously samples the air around it, and passes the air through handheld filtration subsystem 307 which may include conventional portable High Efficiency Particulate Air (HEPA) filters. Particles 102 resulting from the filtering process may then be washed, if required based upon the technology and solution, and the solid samples may be converted into a liquid medium by handheld sampling subsystem 323, such as MesoSystems® BT-550 (under development). The liquid medium is then used for near real time and offline analyses by analysis subsystem 127 to determine if bio-hazardous particles are present. Handheld enclosure 311 surrounds handheld collection subsystem 309 and optionally handheld sampling subsystem 323. Analysis subsystem 127 could also be housed in handheld enclosure 311. Portable particle collector 305 could include a portable model of a conventional particle collector such as that used in mail sortation system 200.

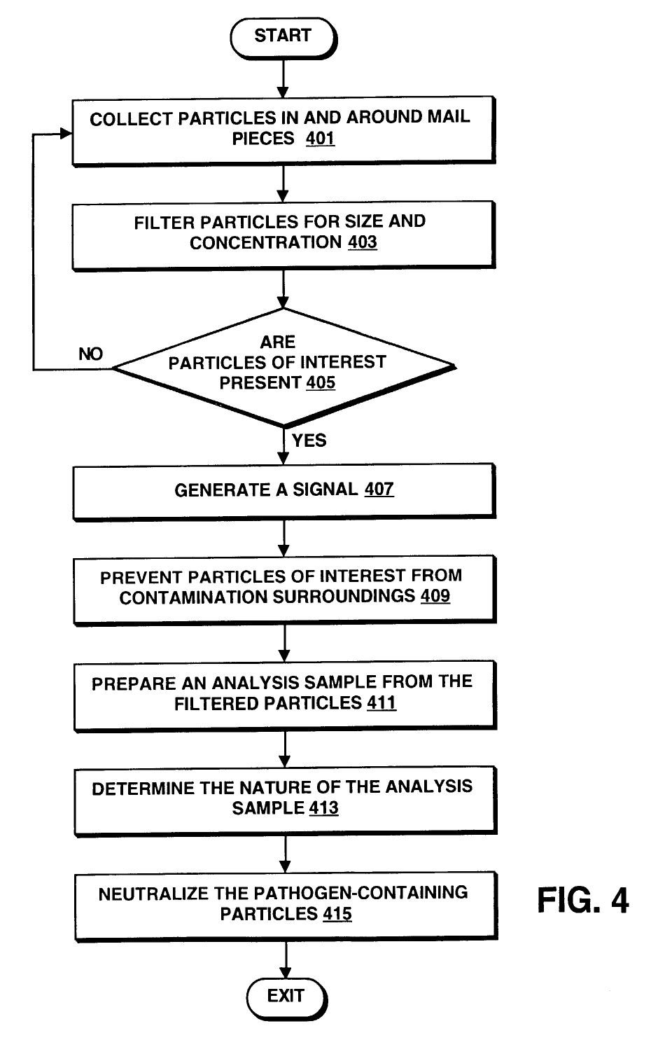

Referring now to FIG. 4, the method of the illustrative embodiment includes the step of collecting particles from the ambient air, possibly through the use of pinch belt pulley assemblies to forcefully eject contaminants and particles from objects (method step 401). The method includes the next steps of filtering particles into the size and concentration of interest (method step 403) and testing filtered particles for the presence of bio-hazardous particles (decision step 405). The method next includes the steps of generating a signal if bio-hazardous particles are present (method step 407) and preventing bio-hazardous particles from contaminating surrounding environment (method step 409). The method next includes the steps of preparing an analysis sample (method step 411), determining the nature of the analysis sample (method step 413), and optionally, isolating and neutralizing the bio-hazardous particles (method step 415).

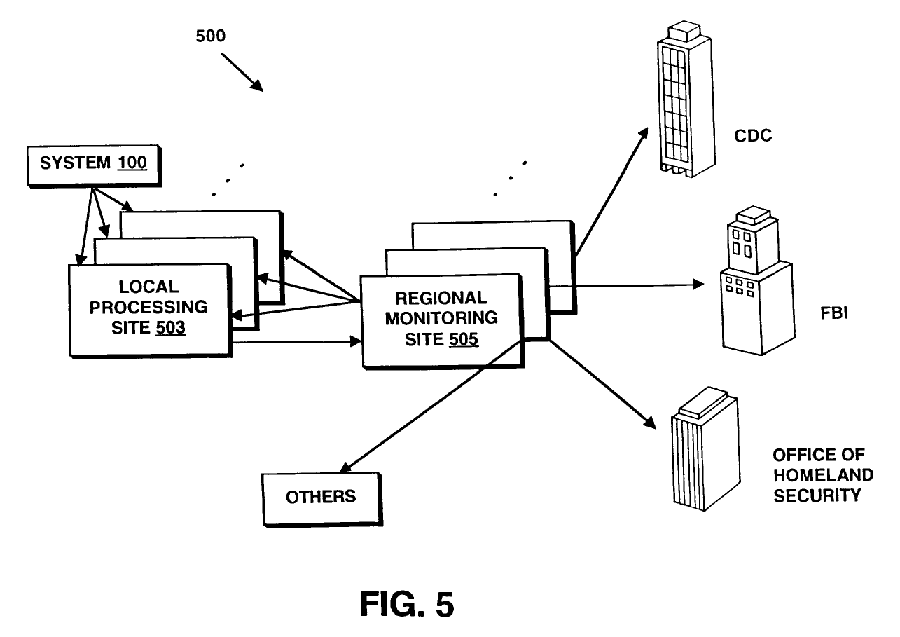

Referring now to FIG. 5, a local, regional, and national monitoring system 500 is depicted in which system 100 is the notification hub for other agencies including receptors of information such as the Centers for Disease Control (CDC), Federal Bureau of Investigation (FBI), and Office of Homeland Security. In this geographically disperse environment, system 100 could transmit contamination events electronically to any one or all of local processing sites 503 for validation, verification, and implementation of plans of action, should system 100 be so configured. Local processing sites 503 could be configured to transmit its contamination events electronically to one or more regional monitoring sites 505, which in turn could communicate these events to receptors such as the CDC, FBI, Office of Homeland Security, and others configured to receive the information. The purpose of creating such a network is to provide remote monitoring of the contamination events and to allow event information to tier up to regional and then national monitoring centers. This event information and data could then be made available to the appropriate federal agencies to allow those agencies to implement the required course of action and appropriate response plans.

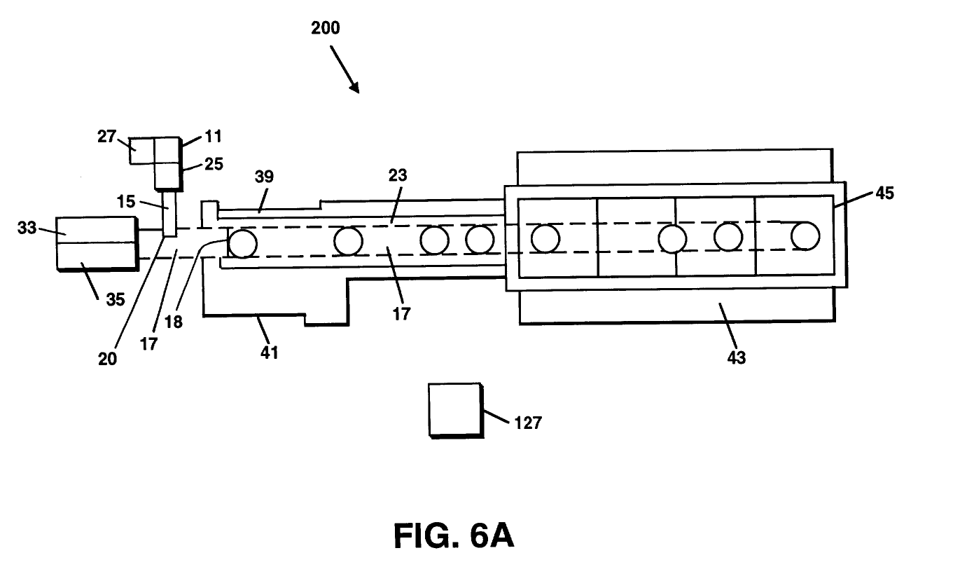

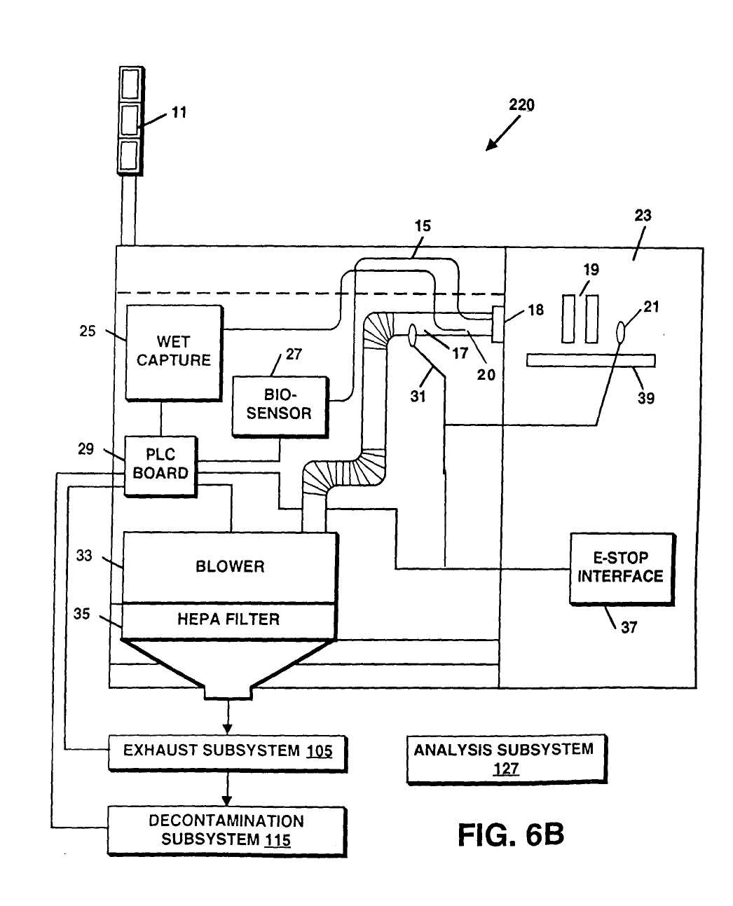

Referring now to FIGS. 6A and 6B, system 220, in which the illustrative embodiment of the control flow 220 for mail sortation is shown. As mail pieces are fed into system 220, through feeder 41, particles are released through normal handling and/or through pinch point pulley assembly 19. Particles are moved through prefilter 18 which allows large particles to pass through the prefilter 18 and exhaust back into the blower/air filtration system 33/35 through simplified hoodless ducting 17 as waste air. Smaller particles enter pitot tube entry 20, into the region in which the particles are tested for contamination. In the illustrative embodiment, the region includes sampling subsystem 123 and triggering subsystem 119 (shown in FIG. 1), embodied in wet capture 25 and biosensor 27/indicator light 11 respectively. After the mail parcels have been fed into the system, they proceed through closed vent/hood 23 on mail transport device 39 towards mail stacker 43 which is enclosed by open vent/hood 45. In general, conventional closed and open vent/hoods 23 and 45, respectively, are custom-fitted to all types of mail transport equipment (i.e. mail transport equipment manufactured by Lockheed Martin, Pitney-Bowes, Bell & Howell, Siemans, etc.) and conventional mail sortation stacker sections 43, pockets, or sort bin destinations typically installed at mail processing facilities as well as commercial pre-sort facilities and mailrooms.

Referring now to specifically to FIG. 6B, conventional programmable logic control (PLC) board 29 sequences operations among the subsystems of system 220, which can be the control flow for mail sortation system 200, mail screening workstation 300, or mail opening workstation 320. Mail flow sensor 21 alerts PLC 29 that mail is traveling along mail transport device 39 towards pinch point pulley assembly 19. PLC 29 activates blower 33 to create a negative air pressure situation that draws air and particles into simplified hoodless ducting 17 through prefilter 18. Larger particles continue on towards blower/air filter 33/35, but smaller particles enter the sample and test systems of the illustrative embodiment of the present invention through pitot tubes 15 at pitot tube openings 20. Shown here are two pitot tubes 15, but there can be any number without changing the scope of the invention. Pitot tube openings 20 are preferably located 0-12 inches from pre-filter 18, and air flow within pitot tubes 15 travels preferably between 100 and 200 cubic feet/minute. Particles travel towards sampling (wet capture 25) and triggering (biosensor 27) simultaneously. Wet capture 25 alerts PLC 29 when a sample is prepared, while biosensor 27 alerts PLC 29 if the particles contain contaminants. When contamination is detected, PLC 29 changes light 11 to indicate a detected condition. Light 11 can be a conventional 3-state indicator, illustratively indicating normal operation, maintenance mode, and fault states. Also, PLC 29 signals E-stop interface 37 to stop the entry of mail to the system (through use of mail flow sensor 21) but continue exhausting particles through the system (through use of air flow sensor 31). PLC 29 signals decontamination subsystem 115 to collect particles exiting from exhaust subsystem 105 and neutralize them.

Continuing to refer to FIG. 6B, biosenser 27 can be a conventional device such as the PSI Bioni® Trigger Mechanism. Air filter 35 can include, in the illustrative embodiment, conventional high rise dust collection filters and dual conventional HEPA filters that capture dust and potentially harmful particles in filter cartridges for later removal and disposal. Prefilter 18, which allows large particles to pass through the filter but captures the smaller particles, is perpetual in nature and does not include filter screens that need to be replaced and have no periodic maintenance needs to be performed on these filter components.

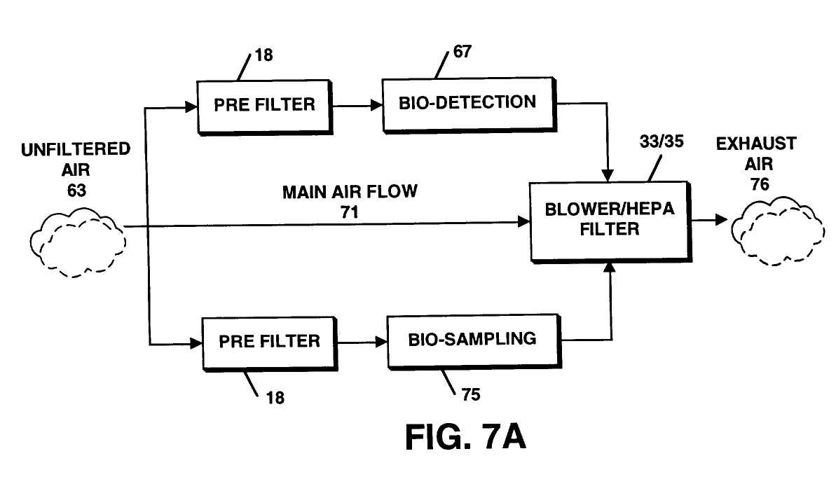

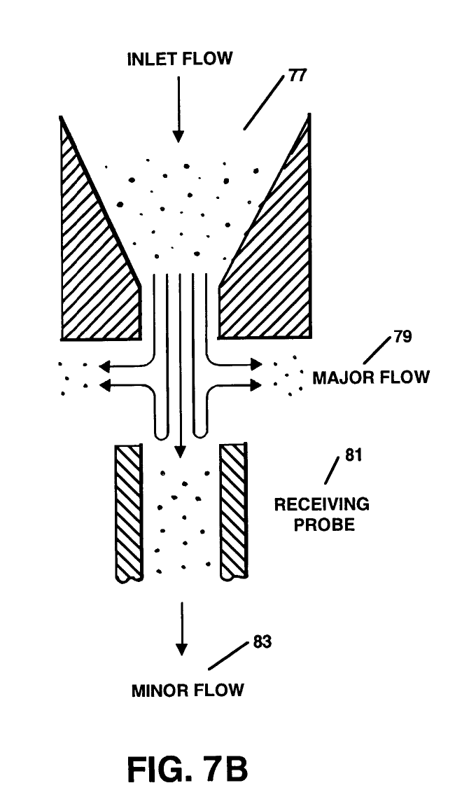

Referring now to FIGS. 7A and 7B, airflow characteristics of the illustrative embodiment of the present invention are shown. The unfiltered air 63 is first captured by a set of pitot tubes, pre-filtered, and transported to the triggering 119 (bio-detection 67) and sampling 123 (bio-sampling 75) subsystems. As previously described, prefilter 18 allows the large particles to pass into main air flow 71, while retaining the smaller particles which are captured by the prefilter 18 at inlet flow 77 (FIG. 7B) and passed to receiving probe 81 (FIG. 7B) (the bio-detection 67 and bio-sampling 75 systems). Air and particles exit receiving probe 81 as minor flow 83, and exit the system through blower/air filter 33/35. The remaining air, main air flow 71, is vented through the ventilation 109 and exhaust 105 subsystems (blower/air filter 33/35) and finally exits the system as exhaust air 76.

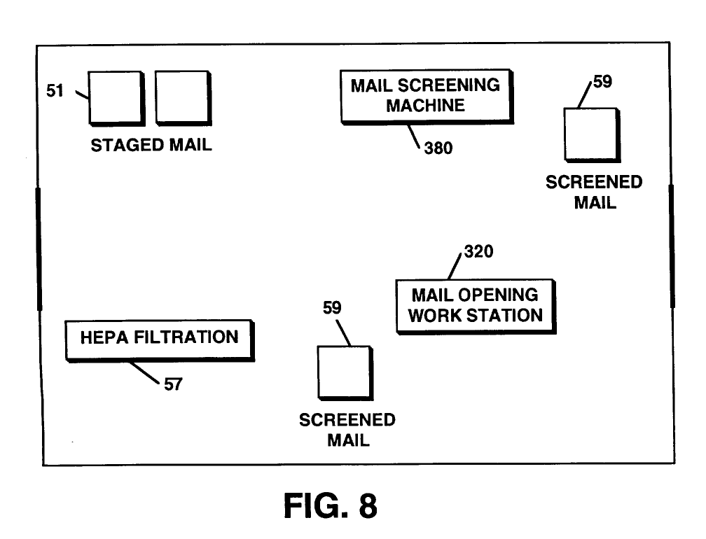

Referring now to FIG. 8, a typical operational environment in shown in which the equipment is installed in and operates in an environment in which a stand alone HEPA ventilation system 57 operates. This type of environment could be used for the mail screening workstation 300 and mail opening workstation 320, or optionally the mail sortation system 200. Operationally, mail enters this environment and is staged in mail staging area 51 for processing. Once the screening process is complete, the screened mail 59 is staged for outbound distribution. Environment 510 is not required but highly recommended due to the nature of screening mail for biological contamination.

Although the invention has been described with respect to various embodiments, it should be realized this invention is also capable of a wide variety of further and other embodiments within the spirit and scope of the appended claims.

What is claimed is:

1. A bio-hazard collection and testing system, comprising:
a collection subsystem for collecting particles in, on and around mail;
a filtration subsystem connected to said collection subsystem for separating biohazardous-sized particles from collected particles for testing and for capturing said collected particles and said bio-hazardous-sized particles;
a triggering subsystem connected to said filtration subsystem for receiving and testing said bio-hazardous-sized particles to determine whether they are bio-hazardous particles, and generating a signal when bio-hazardous particles are detected; and
a sampling subsystem connected to said triggering subsystem for preparing an analysis sample containing said bio-hazardous particles.

2. The system as defined in claim 1 further comprising an analysis subsystem for receiving said analysis sample from said sampling subsystem and determining a composition of said bio-hazardous particles in said analysis sample.

3. The system as defined in claim 1 further comprising an enclosure surrounding at least a part of said collection subsystem, said enclosure creating a barrier between said biohazardous particles and ambient air.

4. The system as defined in claim 1 further comprising an exhaust subsystem operating within said enclosure for forcing said collected particles and said bio-hazardous particles through said filtration subsystem.

5. The system as defined in claim 4 wherein said exhaust subsystem comprises a fan.

6. The system as defined in claim 1 further comprising a decontamination subsystem connected to said filtration subsystem for neutralizing said bio-hazardous particles.

7. The system as defined in claim 1 further comprising a transport/pinch point subsystem for moving mail past said collection subsystem and for forcing particles to be released from the mail.

8. The system as defined in claim 7 wherein said triggering subsystem further comprises a transport subsystem disabling mechanism for disabling said transport/pinch point subsystem when said bio-hazardous particles are detected.

9. The system as defined in claim 1 further comprising a ventilation subsystem for providing airflow communication among said collection subsystem, said triggering subsystem, and said exhaust subsystem, said ventilation subsystem for moving said collected particles and said bio-hazardous particles from said collection subsystem to said exhaust subsystem.

10. The system as defined in claim 1 wherein said triggering subsystem comprises a means for external notification when said bio-hazardous particles are detected.

11. The system as defined in claim 10 wherein said means for external notification comprises:
means for generating a notification signal when said bio-hazardous particles are detected; and
means for routing said notification signal to receptors.

12. The system as defined in claim 1 wherein said collection subsystem further comprises a downdraft mechanism for directing said collected particles away from an operator.

13. The system as defined in claim 1 wherein said filtration subsystem comprises:
a prefilter for separating said bio-hazardous-sized particles from said collected particles; and
a HEPA filter for capturing said collected particles and said bio-hazardous particles.

14. The system as defined in claim 1 wherein said triggering subsystem comprises at least one sensing device for testing said bio-hazardous-sized particles for contamination; and
at least one pitot tube connecting said collection subsystem with said sensing device, said at least one pitot tube for transporting said bio-hazardous-sized particles to said sensing device.

15. The system as defined in claim 1 wherein said sampling subsystem comprises
means for receiving said signal from said triggering subsystem when said triggering subsystem detects said bio-hazardous particles;
at least one sampling device for preparing an analysis sample from said biohazardous particles when said signal is received by said means for receiving; and
at least one pitot tube connecting said collection subsystem with said sampling device, said at least one pitot tube for transporting said bio-hazardous-sized particles to said sampling device.

16. A method for detecting and capturing bio-hazardous particles comprising the steps of:
collecting particles in, on, and around mail;
filtering bio-hazardous-sized particles from collected particles;
testing said bio-hazardous-sized particles for the presence of bio-hazardous particles;
generating a signal when said bio-hazardous particles are detected;
preparing an analysis sample when said signal is generated;
filtering said collected particles from surrounding air; and
exhausting filtered air.

17. The method as defined in claim 16 further comprising the steps of:
preventing said bio-hazardous particles from contaminating surroundings when said bio-hazardous particles are detected;
determining the nature of the bio-hazardous particles; and
neutralizing the bio-hazardous particles.

18. A method for detecting pathogens in the mail stream comprising the steps of:
transporting mail near a particle collection area;
subjecting the mail to pressure to release particles;
collecting particles in, on, and around the mail;
separating bio-hazardous-sized particles from collected particles;
exhausting collected particles through a filter;
testing bio-hazardous-sized particles for bio-hazardous particles;
stopping said transporting of mail if said bio-hazardous particles are detected;
illuminating an alarm light if said bio-hazardous particles are detected;
preparing an analysis sample of said bio-hazardous particles;
exhausting said bio-hazardous-sized particles through said filter; and
neutralizing said bio-hazardous particles if said bio-hazardous-sized particles are detected.

19. The method as defined in claim 18 further comprising the step of:
analyzing said analysis sample.

20. The method as defined in claim 18 further comprising the step of:
notifying receptors when said bio-hazardous particles are detected.
